(19) United States
(12) Patent Application Publication
(10) Pub. No.: US 2002/0007194 A1
(43) Pub. Date: Jan. 17, 2002

(54) Devices for closing off an aneurysm or the like in a blood vessel such as an artery

(76) Inventor: Nicolas Plowiecki, Montmorency (FR)

Correspondence Address: SUGHRUE, MION, ZINN, MACPEAK & SEAS, PLLC, Suite 800, 2100 Pennsylvania Avenue, N.W., Washington, DC, 20037-3202, US

(21) Appl. No.: 09/739,320

(22) Filed: Dec. 19, 2000

(30) Foreign Application Priority Data
Jul. 13, 2000 (FR) 9900121

(51) Int. Cl. A61M 25/10

(52) U.S. Cl. 606/194

(57) ABSTRACT

The present invention relates to devices for closing off an aneurysm 1 or the like in a blood vessel 2 such as an artery. The device of the invention comprises a catheter 10 suitable for sliding in translation in the blood vessel 2, a set of rings 11, 12, 13, ... substantially all of the same diametral section, each ring being suitable for deploying radially between first and second section values that are respectively less than and substantially equal to the inside section of the blood vessel, the set of rings 11-13 being engaged on the catheter 10 when the rings are in the configuration in which their section has the first value, the rings being substantially touching, a flexible and elastic sheath 15 surrounding and touching the set of rings 11-13, and means 16 for controlling radial deployment of the rings to bring their section from the first value to the second value. The device is particularly suitable for treating cerebral aneurysms.

The present invention relates to devices for closing off an aneurysm or like in a blood vessel such as an artery, i.e. devices which are known by the person skilled in the art as “stents” or “endoprostheses”, and more particularly it relates to those which are suitable for treating cerebral aneurysms.

### BACKGROUND OF THE INVENTION

Devices are already known for closing off aneurysms or the like to be closed in a blood vessel such as an artery.

By way of example, such devices are described in: US-A-5 449 373, US-A-5 195 984, EP-A-0 795 304, EP-A-0 832 618, and WO 96/18359. In general they are constituted by: a catheter suitable for sliding in translation in the blood vessel; a ring suitable for deploying radially between first and second section values that are respectively less than and substantially equal to the inside section of the blood vessel, the ring being engaged on the catheter in the configuration in which its section has the first value; and means for controlling radial deployment of the ring to bring its section from its first value to its second value.

In many cases that device is satisfactory. However, it can suffer from drawbacks when the blood vessels have numerous bends of small radius, e.g. arteries in the brain. Under such conditions, since the ring of the device is too long and too rigid, it cannot always be brought to the aneurysm since it cannot curve sufficiently to pass certain bends in the arteries.

In an attempt to mitigate that drawback, points of weakness have been provided over the entire length of the ring and at relatively close regular intervals, making it possible, in theory, for the ring to fold more easily and thus to pass small-radius bends. However, it is then observed that if the number of bends is large and if they are very sharp, then the ring weakens at the points of weakness until it finally breaks.

### OBJECTS AND SUMMARY OF THE INVENTION

Thus, an object of the present invention is to provide a device for closing an aneurysm or the like in a blood vessel such as an artery, mitigating the drawbacks of similar prior art devices mentioned above.

More precisely, the present invention provides a device for closing off an aneurysm or the like in a blood vessel such as an artery, the device comprising:

a catheter suitable for sliding in translation in the blood vessel;

a set of rings substantially all of the same diametral section, each ring being suitable for deploying radially between first and second section values that are respectively less than and substantially equal to the inside section of the blood vessel, the set of rings being engaged on the catheter while the rings are in the configuration in which their section has the first value, the rings being substantially touching;

a flexible and elastic sheath surrounding and touching the set of rings engaged on the catheter; and

means for controlling the radial deployment of the rings to bring their section from the first value to the second value.

### BRIEF DESCRIPTION OF THE DRAWINGS

Other characteristics and advantages of the present invention appear from the following description given by way of non-limiting illustration and with reference to the accompanying drawings, in which:

FIGS. 1 and 2 are diagrammatic section views showing an embodiment of a device of the invention for closing off an aneurysm or the like in a blood vessel such as an artery, FIG. 1 showing the device in its initial configuration and FIG. 2 showing the same device in its configuration which enables the aneurysm to be treated;

FIGS. 3 to 5 are illustrative diagrams showing three possible embodiments of component elements of the device of the invention; and

FIG. 6 is an overall diagram for explaining how the device of the invention is used.

### MORE DETAILED DESCRIPTION

The Applicant wishes to emphasize that these figures merely show examples of the subject matter of the invention, and that other embodiments can exist that satisfy the definition of the invention.

The Applicant also specifies that when the subject matter of the invention is defined as having “at least one” element with a given function, then the embodiment described may have more than one or such element.

The Applicant also specifies that if the embodiment of the invention as shown has a set of elements of identical function, and if in the description it is not stated that the subject matter of the invention must necessarily have some particular number of such elements, then the subject matter of the invention can be defined as having “at least one” of these elements.

It is also specified that the same references are used in all the figures to designate the same elements whatever the figure in which they appear and whatever the way in which they are shown.

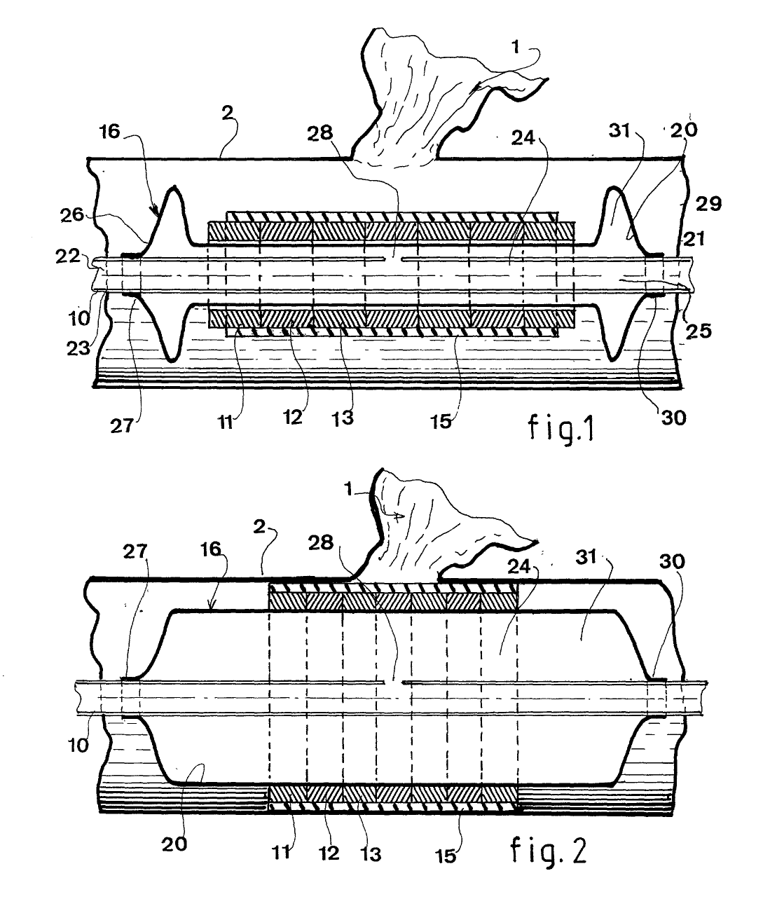

The device shown in FIGS. 1 and 2 serves to close off an aneurysm 1 or the like in a blood vessel 2 such as an artery, and it is particularly suitable for treatment of cerebral aneurysms.

It comprises a catheter 10 shaped to be suitable for sliding in translation in the blood vessel 2 regardless of the shape of the blood vessel, i.e. it is suitable for easily passing bends of any radius whatsoever. Catheters presenting this characteristic are well known per se and are not described in greater detail herein.

The device also has a set of rings 11, 12, 13, ... substantially all of the same diametral section, each ring being suitable for deploying radially between a first section value, FIG. 1, and a second section value, FIG. 2, which values are respectively less than and substantially equal to the inside section of the blood vessel. This set of rings 11-13 is engaged on the catheter 10 while the rings are in the configuration in which their section has the first value and in such a manner that the rings are substantially touching.

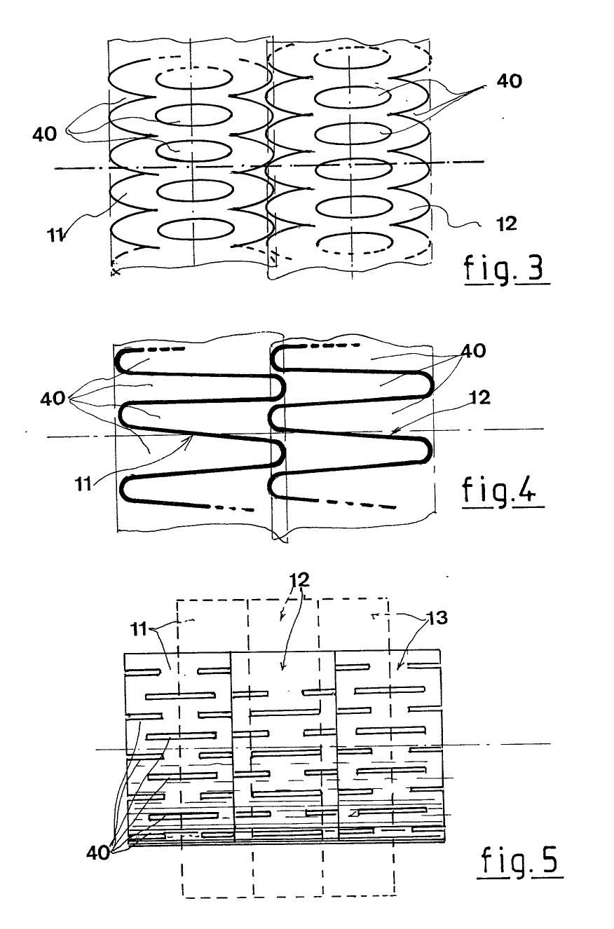

FIGS. 3 to 5 are fragmentary diagrams showing three possible embodiments of the rings, enabling them to satisfy the above-mentioned conditions.

With reference to these FIGS. 3 to 5, the rings 11-13 are made of a metal, e.g. steel, gold, or platinum, or else of a material known under the trade name “NITINOL”, and they have cut-outs 40 enabling them to be deployed radially. In conventional manner, by exerting traction and/or thrust in a radial direction on such rings, the cut-outs are deformed, thereby increasing the diameter of the rings, it being understood that this increase in diameter itself gives rise to a shortening in the length of the rings, as can be seen in FIG. 5 in which one particular possible embodiment of the rings 11-13 is shown, with continuous lines showing them in their initial configuration in which their section has the first value, and with dashed lines showing them in their deployed configuration in which their section has the second value.

These cut-outs can be made in various ways, for example by means of a laser beam as is the case for the embodiments of FIGS. 3 and 5, or by folding a wire into a closed zig-zag shape as is the case for the embodiment of FIG. 4, the gaps between the loops in the zig-zag shape constituting the above-defined cut-outs 40.

Furthermore, as shown in FIGS. 3 and 4, the rings 11-13 can have cut-outs 40 which are lateral so that during initial positioning around the catheter 10, the substantially touching rings can also interfit one in another for the purpose of ensuring that radial deployment thereof does not give rise to excessive reduction in the length of the set of rings.

In all cases, it is nevertheless preferable for the rings to be generally circularly cylindrical in shape and for their width to be less than their diameter, even in their initial configuration in which their section has the first value, see FIGS. 3, 4, and 5.

It is nevertheless emphasized that the three embodiments of rings shown in FIGS. 3 to 5 are merely possible examples, and other examples can naturally also exist.

The device also has a flexible and elastic sheath 15 mounted on the outside surface of the rings 11-13, surrounding them and in contact therewith, and means 16 for controlling radial deployment of the rings to bring their section from the first value to the second value.

In a preferred embodiment, these means 16 for controlling radial deployment of the rings to bring their section from the first value to the second value are constituted by an inflatable balloon 20 disposed between the inside wall of the set of rings 11-13 and the outside wall of the catheter 10, and by means 22 for controlling inflation of the balloon.

The means 22 for controlling inflation of the balloon are advantageously constituted by a duct 23 opening out into the balloon via one of its ends 24, its other end being suitable for being connected to the source of fluid under pressure, and by an orifice 28 formed in the wall of the duct 23 to put the inside 25 of the duct into communication with the inside 31 of the balloon 20.

To enable the rings to be put into place properly, as explained below, it is also advantageous for the catheter 10 to pass through the wall 26 of the balloon 20 via at least one sealed passage 27 and for the duct 23 to be inside the catheter 10. In this way, the sealed passage 27 also constitutes a means for securing one point of the balloon 22 to the catheter.

Nevertheless, it is even more advantageous for the catheter 10 to pass through the wall 26 of the balloon 20 via two sealed passages 27, 30 at two substantially opposite points in the wall of the balloon, as shown in FIGS. 1 and 2. In this way, the balloon is held securely and cannot move along the catheter. As a result its position is always well defined relative to the catheter 10. Advantageously, the distance between these two sealed passages 27, 30 is greater than the length of the set of rings 11-13 in its initial configuration, so that the balloon projects beyond each end of said set of rings.

In an advantageous embodiment, the sheath 15 is made of a synthetic material, for example polyurethane, polyamide, etc. Furthermore, as shown in FIG. 1, the length of the sheath 15 lies between the length of the set of rings in its initial configuration in which the section of the rings has its first value and the length of the set of rings minus one ring in the same configuration. In this way, all of the rings 11-13 are held securely against one another when they are inserted together with the catheter into the blood vessel, as explained below.

The device described above and shown in FIGS. 1 to 6 is used and operates as follows:

The device is initially assembled as shown in FIG. 1, i.e. with the rings 11-13 touching in the configuration in which their section has the first above-defined value, disposed around the balloon 20 mounted in association with the catheter 10, and with the sheath 15 surrounding the rings, exerting a small amount of resilient pressure on the outside wall thereof.

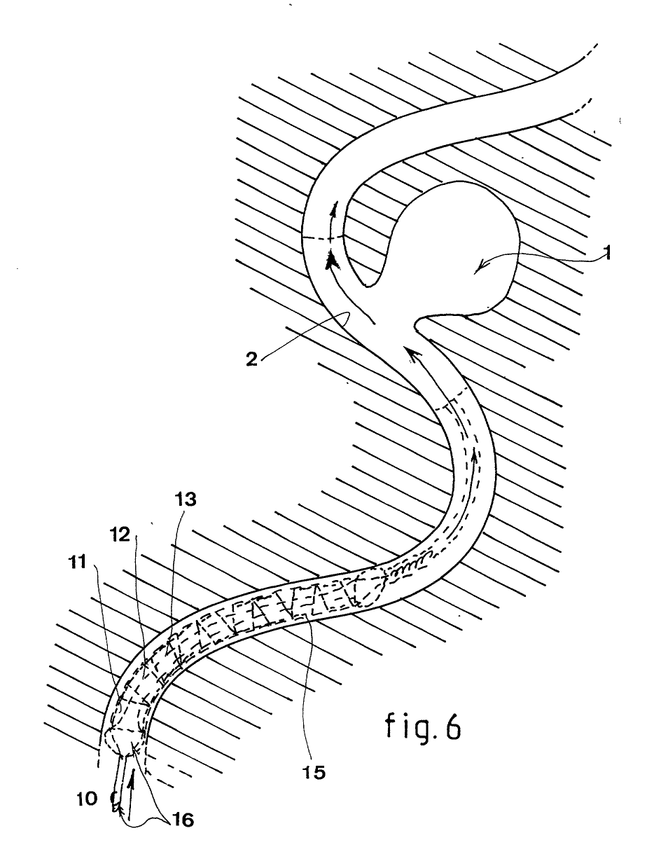

In a manner known to the person skilled in the art, the catheter is inserted into the blood vessel which is to be treated, e.g. a cerebral artery 2 which has an aneurysm 1, FIG. 6, starting from a location of the blood vessel that is easily accessible. The catheter is inserted via its end carrying the balloon 20 and the set of rings 11-13 with the sheath 15. It is then forced along the artery until the set of rings comes into register with the aneurysm 1. It should be observed that this movement of the catheter in translation is possible, even round bends of small radius, because the rings can pivot relative to one other about axes that are substantially perpendicular to the longitudinal axis of the catheter 10, while still being held by the sheath.

It is possible for the rings to be held because the sheath is flexible and elastic. The set of rings can zig-zag like a snake slithering, the elasticity of the sheath making possible simultaneously for the rings to rotate relative to one another as described above and for them to return to their initial position when a bend has been passed and the set of rings is again in a rectilinear portion of artery.

Once the set of rings has come into register with the aneurysm 1, the balloon is inflated. As it inflates, it exerts thrust on the inside wall of the rings which deploy radially until they take up their second section value, FIG. 2, thereby closing off or obstructing the aneurysm 1.

It should be observed that the length of the sheath is preferably defined so that it does not extend beyond each end of the set of rings, neither in its initial configuration so as to avoid impeding progress of the catheter 10 in the artery 2, nor once the rings are deployed, FIG. 2.

It should be observed that the decrease in the length of the set of rings 11-13 during radial deployment thereof is minimized for rings which, in their initial configuration, interfit in one another, e.g. as shown in FIGS. 3 and 4.

When the rings are deployed radially, the balloon is deflated and the catheter is withdrawn from the artery. Only the rings 11-13 surrounded by the sheath 15 closing off the aneurysm are left in place.

Naturally, the ability of the rings to withstand deformation is greater than the elastic force generated by the sheath material so that when the rings are deployed and the balloon deflated, the banded elastic sheath does not deform the rings to return them to their initial configuration.

1. A device for closing off an aneurysm or the like in a blood vessel such as an artery, the device comprising:
a catheter suitable for sliding in translation in the blood vessel;
a set of rings substantially all of the same diametral section, each ring being suitable for deploying radially between first and second section values that are respectively less than and substantially equal to the inside section of the blood vessel, the set of rings being engaged on the catheter while the rings are in the configuration in which their section has the first value, the rings being substantially touching;
a flexible and elastic sheath surrounding and touching the set of rings engaged on the catheter; and
means for controlling the radial deployment of the rings to bring their section from the first value to the second value.

2. A device according to claim 1, wherein the means for controlling radial deployment of the rings to bring their section from the first value to the second value are constituted by an inflatable balloon disposed between the inside wall of the set of rings and the outside wall of the catheter, and by means for controlling inflation of the balloon.

3. A device according to claim 2, wherein the means for controlling inflation of the balloon are constituted by a duct opening out into the balloon via one of its ends, its other end being suitable for connection to a source of fluid under pressure, and by an orifice made through the wall of the duct to put the inside of the duct into communication with the inside of the balloon.

4. A device according to claim 3, wherein the catheter passes through the wall of the balloon via at least one sealed passage, the duct being in the catheter.

5. A device according to claim 1, wherein the sheath is made of a synthetic material.

6. A device according to claim 1, wherein the rings are made of a metal and wherein the rings have cut-outs to enable them to deploy radially.

7. A device according to claim 1, wherein the length of the sheath lies between the length of the set of rings in the configuration in which their section has the first value and the length of the set of rings minus one ring in the same configuration.

8. A device according to claim 4, wherein the catheter passes through the wall of the balloon via two sealed passages.

9. A device according to claim 8, wherein the distance between the two sealed passages is greater than the length of the set of rings.

10. A device according to claim 1, wherein the rings interfit in one another.

11. A device according to claim 1, wherein the rings are generally circularly cylindrical in shape, their width being less than their diameter, even in their initial configuration in which their section has the first value.
